(19) United States
(12) Patent Application Publication
(10) Pub. No.: US 2003/0074006 A1
(43) Pub. Date: Apr. 17, 2003

(54) Medical device with enhanced guide capabilities

(76) Inventors: David H. Mowry, Waconia, MN (US); Michael Gregory O'Connor, Lino Lakes, MN (US); Sean Patrick Skubitz, Shoreview, MN (US)

Correspondence Address: MERCHANT & GOULD PC, P.O. BOX 2903, MINNEAPOLIS, MN, 55402-0903, US

(21) Appl. No.: 09/976,258

(22) Filed: Oct. 11, 2001

(51) Int. Cl. A61F 11/00

(52) U.S. Cl. 606/108

(57) ABSTRACT

The present invention relates to a medical device including two parallel tubes attached to each other, one of which is shorter in length and receives a guide snare for directing the path of the catheter within a patient's blood vessels and into the patient's heart. The present invention further relates to a method of using a catheter comprising two tubes, whereby a guide snare is anchored to a shunt within a patient's heart and extends out of the patient's body, one of the tubes of the catheter being placed about the guide snare and the catheter being advanced into the patient's heart along the guide snare.

### FIELD OF THE INVENTION

The present invention relates to medical catheters for performing diagnostic procedures and other intervention procedures using a catheter placed into a patient's body.

### BACKGROUND OF THE INVENTION

The placing of artificial shunts or other durable passageways in the heart wall to connect heart chambers containing oxygenated blood with coronary arteries is known. These devices and the techniques for placing them in the heart are described in detail in U.S. Pat. No. 5,944,019, issued Aug. 31, 1999, which is hereby incorporated by reference. Collectively, in this application, these devices, including artificial shunts and other durable passageways will be referred to solely as shunts. Such shunts typically are placed in the wall of the heart to allow oxygenated blood to flow into a partially or completely occluded coronary artery as an alternative to more traditional or conventional vein graft coronary arterial bypass procedures. What is needed are effective techniques for accessing the shunts for diagnostic reasons or other reasons.

### SUMMARY OF THE INVENTION

The present invention relates to a diagnostic catheter including two parallel tubes attached to each other, one of which is shorter in length and receives a guide snare for directing the path of the catheter to a location within a patient's body. The present invention further relates to a method of using a catheter comprising two tubes, whereby a guide snare is anchored to a shunt within a patient's heart and extends out of the patient's body, one of the tubes of the catheter being placed about the guide snare and the catheter being advanced into the patient's heart along the guide snare.

A variety of advantages of the invention will be set forth in part in the description that follows, and in part will be apparent from the description, or may be learned by practicing the invention. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of the description, illustrate several aspects of the invention and together with the description, serve to explain the principles of the invention. A brief description of the drawings is as follows:

FIG. 1 is a view of a distal end of a catheter according to the present invention.

FIG. 2 is a cross-sectional view of the distal end of the catheter in FIG. 1.

FIG. 3 is a schematic illustration with a heart in partial cutaway of a guide snare extending through a femoral artery into the left ventricle of a patient.

FIG. 4 is a close up view of the heart of FIG. 3, showing the guide snare within the left ventricle of the patient anchored to a stent in the heart wall.

FIG. 5 is a view of a distal end of the catheter of FIG. 1 within the left ventricle of the heart of FIG. 4.

FIG. 6 is a view of a heart of in partial cutaway with a distal end of a guide snare within the left ventricle of the heart.

FIG. 7 is a view of the heart in FIG. 6 with a guide snare including a distal end anchor extending from the distal end of the catheter.

FIG. 8 is a view of an alternative catheter according to the present invention.

FIG. 9 is a view of an alternative catheter according to the present invention.

### DETAILED DESCRIPTION

Reference will now be made in detail to exemplary aspects of the present invention which are illustrated in the accompanying drawings. Wherever possible, the same reference numbers will be used throughout the drawings to refer to the same or like parts.

Cardiac catheterization is the process of inserting a catheter, typically through a major blood vessel, such as a femoral artery 12, into a heart 26 of a patient. Cardiac stenting is the process of providing a stable passage in the form of a stent or shunt 30 through heart wall 32 for blood to flow out of a chamber, such as left ventricle 22, of heart 26 into a coronary artery, such as left anterior descending coronary artery 38. Cardiac catheterization via a femoral artery is may be used to position a diagnostic catheter, such as catheter 100 within heart 26 proximate shunt 30. Other methods of cardiac catheterization for diagnostic purposes may be found in jointly owned and copending application entitled, Interventional Diagnostic Catheter and A Method for Using a Catheter to Access Artificial Cardiac Shunts, filed Aug. 15, 2001, Attorney Docket No. 11587.34US01, which is incorporated herein by reference.

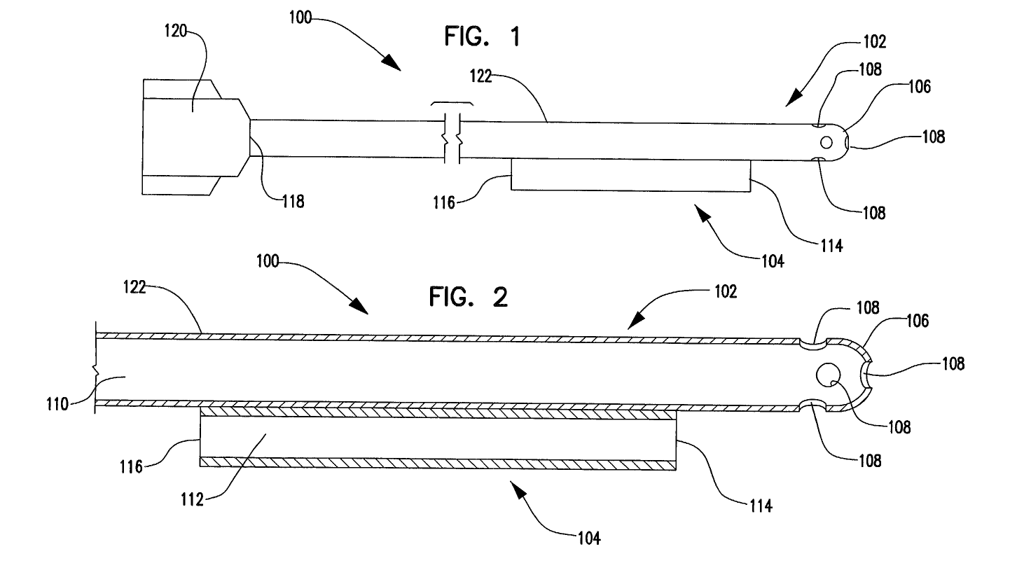

Referring now to FIGS. 1 and 2, a catheter 100 is shown including a diagnostic tube 102 and a guide tube 104. Diagnostic tube 102 is a hollow tube and includes a distal end 106 having one or more openings 108 allowing fluid communication with an interior space 110. Alternatively, distal end 106 may have an open end or may incorporate a basket. Attached to diagnostic catheter 102 proximate distal end 106 is guide tube 104. Guide tube 104 is also a hollow tube having an interior passageway 112 extending from distal end 114 to proximal end 116. Length of guide tube 104 is between 2.5 cm and 8 cm, preferably 8 cm. The length of guide tube 104 will depend on the flexibility of the material from the which guide tube 104 is formed and sharpness of the bends about which the tube will be directed. The diameter of guide tube 104 is sufficient to have an inner diameter that will accept and slide along a guide snare 130, as shown below. Commonly, these snares are approximately 4 French in size. As shown, distal end 114 and proximal end 116 of guide tube 104 may be flat-faced, or more have a truncated conic shape to aid insertion through blood vessels and heart valves.

Length of diagnostic catheter 102 is sufficient to allow the insertion of distal end 106 into heart 26, as shown below, such that hub 120 located proximate proximal end 118, remains outside of the patient's body. The desired length may vary based on the entry site of the catheter into the patient's blood vessels and the physical size of the patient. Hub 120 is adapted to allow tools, such as snares, or diagnostic fluids, such as radio-opaque dyes, to be injected into interior 110 to flow out openings 108 into left ventricle 22.

As shown, guide tube 104 may be an integral part of diagnostic tube 102. Alternatively, guide tube 104 may be a separate tube which is bonded or otherwise attached to outer surface 122 of diagnostic tube 102. Guide tube 104 as shown is a continuous tube so that a guide snare 130 received within interior passageway 112 by inserting distal end 134 into interior passageway openings at distal end 114.

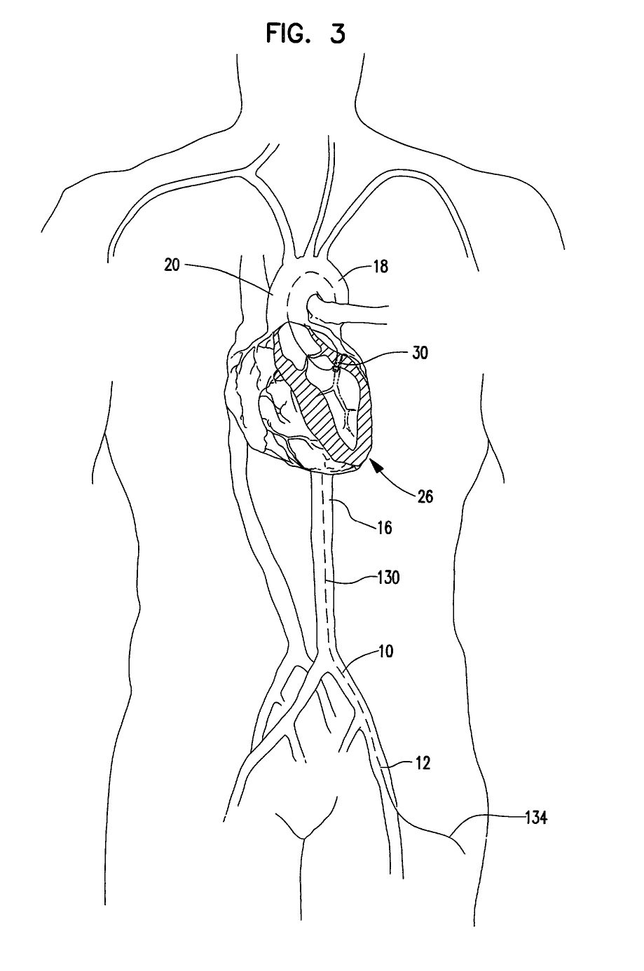

Referring now to FIGS. 3 through 5, a guide snare 130 is shown removably attached by an anchor 132 to an end of shunt 30 extending into left ventricle 22. Guide snare 130 enters a femoral artery 10 at an insertion site 12 in a patient's groin. From there, guide snare 130 extends up descending aorta 16 over aortic arch 18 and into ascending aorta 20. Passing through ascending aorta 20, guide snare 130 crosses aortic valve 24 to enter left ventricle 22. At the distal end of guide snare 130 is anchor 132 which releasably holds guide snare 130 to shunt 30 within left ventricle 22. Shunt 30 provides a stable passageway for oxygenated blood to travel from within left ventricle 22 through heart wall 32 directly into coronary artery 38. Guide snare 130 can be directed placed on shunt 30 through the use of a guide catheter 43, discussed below and such as that disclosed in jointly owned and co-pending application entitled, Interventional Diagnostic Catheter and A Method for Using a Catheter to Access Artificial Cardiac Shunts, filed Aug. 15, 2001, Attorney Docket No. 11587.34US01, which is incorporated herein by reference.

With guide snare 130 in place within left ventricle 22, diagnostic tube 102 can be advanced into left ventricle 22, with distal end 106 positioned proximate shunt 30. To accomplish this, guide snare 130 is inserted within interior opening 112 of guide tube 104. This insertion may be accomplished by threading proximal end 134 of guide snare 130 through distal end 114 of guide tube 104 and extending guide snare 130 through proximal end 116.

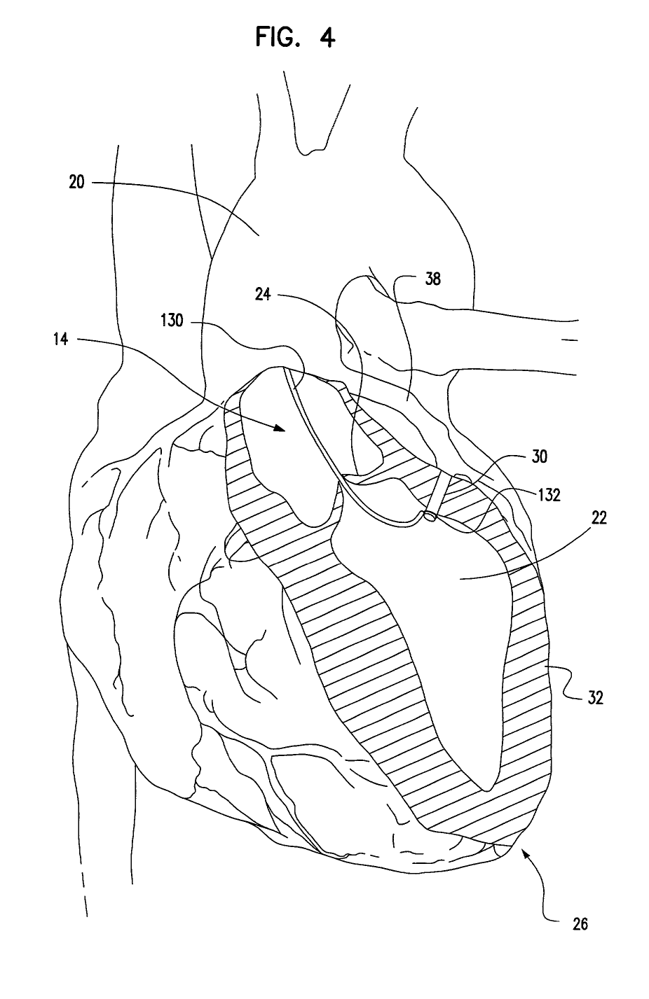

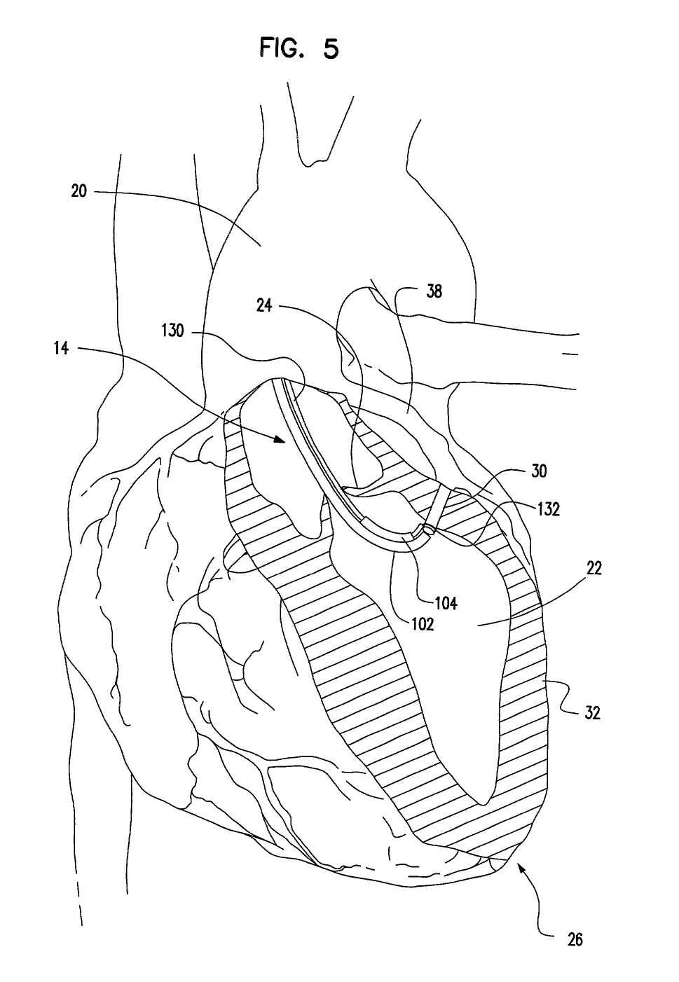

FIG. 4 shows guide snare 130 releasably attached to shunt 30 by anchor 132 at the distal end of guide snare 130. In this position, guide snare 130 may be used as a guide for directing other tools or diagnostic devices such as catheter 100 into left ventricle 22, such that the distal end of such a device may be located proximate shunt 30. FIG. 5 shows guide tube 104 positioned about guide snare 130 within left ventricle 22. With guide tube 104 is located proximate distal end 106 of diagnostic tube 102, advancing guide tube 104 toward anchor 132 carries distal end 106 to a location proximate shunt 30. Alternatively, distal end 106 of catheter 100 may be advanced along guide snare 130 and directed into shunt 30, or placed over shunt 30 if distal end 106 comprises a bracket. In this position, material may be inserted through hub 120, into interior space 110 of diagnostic tube 102 and into left ventricle 22. The material injected may include but not be limited to radio-opaque dyes to aid cardiac imaging to determine blood flow through shunt 30 into coronary artery 38.

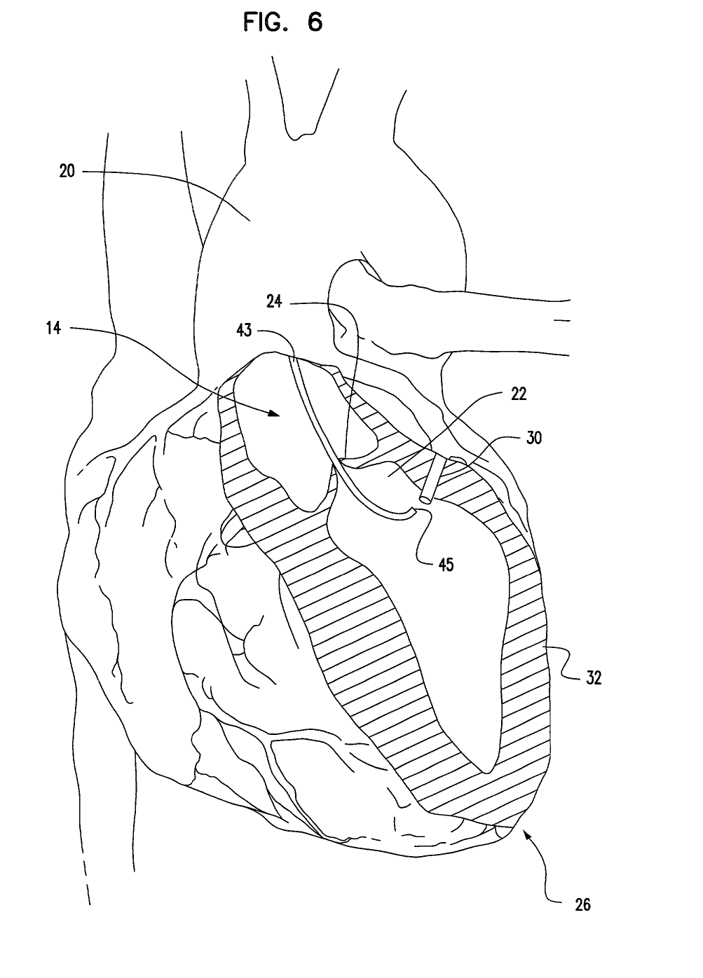

Referring now to FIGS. 3 through 7, a method of positioning diagnostic catheter 100 within left ventricle 22 is shown. The reach this position, an alignment catheter 43 is inserted at insertion site 12 in femoral artery 10 in a patient's groin. Alignment catheter 43 is then advanced retrograde through femoral artery 10, descending aorta 16, over aortic arch 16, and through ascending aorta 20, similar to the path of guide snare 130 shown in FIG. 3. Distal end 45 of alignment catheter 43 is then advanced through aortic valve 24 to arrive at a position within the left ventricle shown in FIG. 6.

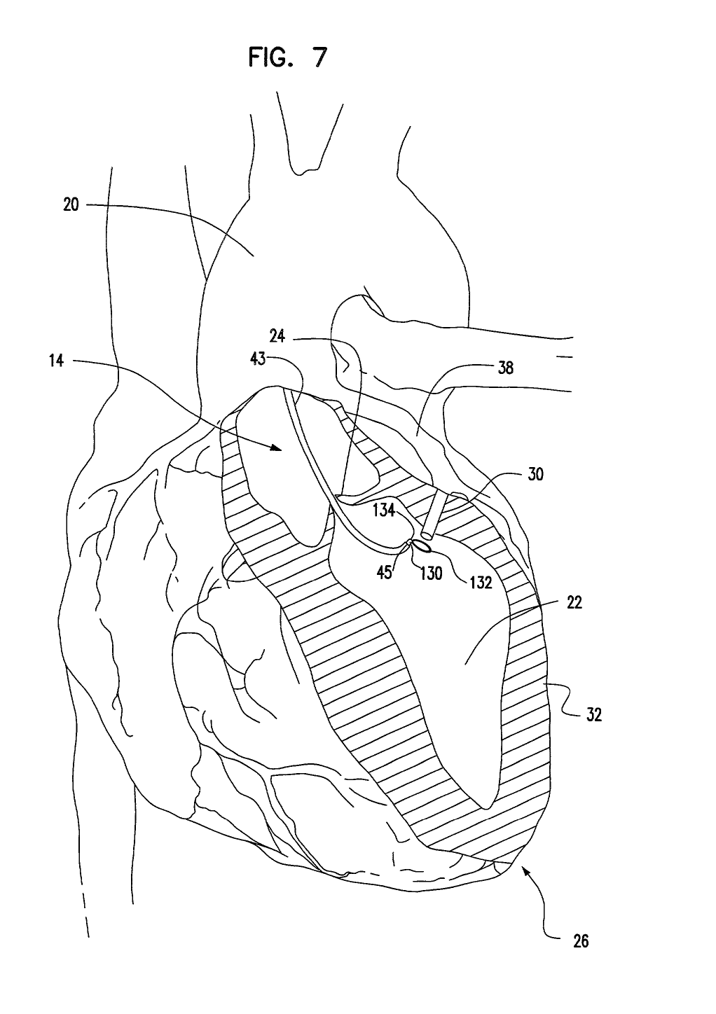

Alignment catheter 43, as disclosed in jointly owned and co-pending application entitled, Interventional Diagnostic Catheter and A Method for Using a Catheter to Access Artificial Cardiac Shunts, filed Aug. 15, 2001, Attorney Docket No. 11587.34US01, which is incorporated herein by reference, is a hollow catheter allowing items from within the catheter to be passed out distal end 45. In FIG. 7, distal end 134 of guide snare 130 with anchor 132 is extended beyond distal end 45 into left ventricle 22. Anchor 132 shown is a simple lasso-type snare. Alternatively, other known snare types suitable for releasably attaching to shunt 30 and anchoring guide snare 130 may be used. Anchor 132 is then extending to lay about shunt 30. Anchor 132 is then drawn snugly about shunt 30 to secure guide snare 130 within left ventricle 22. Alignment catheter 43 can then be withdrawn from heart 26 and from the patient's body, leaving guide snare 130 anchored to shunt 30, as shown in FIGS. 3 and 4.

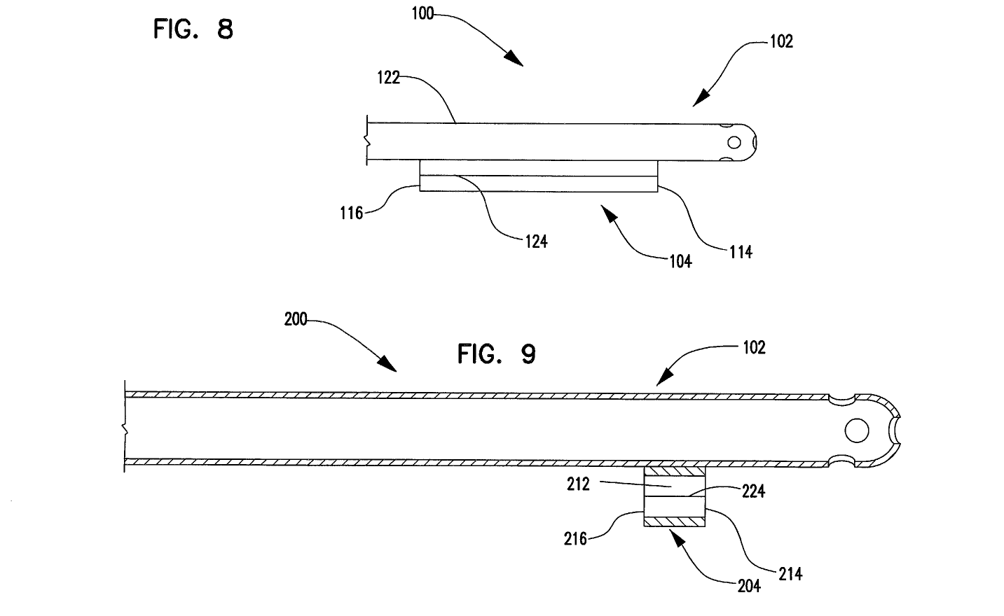

Alternatively, guide tube 104 may include a lengthwise split 124, extending from distal end 114 to proximal end 116, split 124 being normally closed. Guide tube 104 may then be made of a resilient deformable material that will allow guide snare 130 to be snapped into guide tube 104 at some location closer to insertion site 12 than proximal end 134. Once guide snare 130 has been inserted into interior 112, split 124 will return to its normal closed position. This alternative embodiment is shown in FIG. 8. A further alternative embodiment of a catheter 200 according to the present invention is shown in FIG. 9. Catheter 200 includes a first hollow tube 102, as described above, and a guide ring 204. Guide ring 204 is attached to first hollow tube 102 proximate distal end 106, and provides an opening 212 for receiving guide snare 130. Guide ring 204 also includes a split 224 allowing guide snare 130 to be placed within opening 212 at a location other than distal end 134.

Diagnostic catheter 100 may also be used to guide medical instruments to regions of the body other than the heart. Any location within the body providing an adequate support for anchor 132 may be accessed with catheter 100 using the apparatus and methods disclosed herein. It is anticipated that guide tube 14 and guide ring 204 may be adapted to other medical devices for use within a patient's body beyond the catheters disclosed herein and similar methods may be used to position these devices.

Having described preferred aspects and embodiments of the present invention, modifications and equivalents of the disclosed concepts may readily occur to one skilled in the art. However, it is intended that such modifications and equivalents be included within the scope of the claims which are appended hereto.

What is claimed is:

1. A catheter comprising a first hollow tube and a substantially parallel second hollow tube:
the first hollow tube having a distal end, a proximal end, a length and a lumen connecting the distal end to the proximal end;
the second hollow tube having a distal end, a proximal end, a length and a lumen connecting the distal end to the proximal end, the distal end and the proximal end each including an opening into the lumen;
the length of the second hollow tube being substantially less then the length of the first hollow tube;
the second hollow tube being attached to the first hollow tube such that the distal end of the second hollow tube is proximate the distal end of the first hollow tube; and
the openings and lumen of the second hollow tube sized to receive a guide snare.

2. The catheter of claim 1, wherein the second hollow tube includes a lengthwise split through a wall of the second hollow tube from distal end to proximal end, the split normally closed, the second hollow tube made of material sufficiently deformable which will allow the guide snare to be inserted through the split into the lengthwise lumen of the second hollow tube and sufficiently resilient for the split to close after the guide snare is inserted.

3. The catheter of claim 1, wherein the second hollow tube is between 2.5 cm and 8 cm in length, preferably 8 cm.

4. The catheter of claim 1, wherein the openings and the lumen of the second hollow tube are sized to permit free passage of a 4 French size guide snare.

5. The catheter of claim 1, wherein the second hollow tube is integral with the first hollow tube.

6. The catheter of claim 1 wherein the second hollow tube is distinct from the first hollow tube and the second hollow tube bonded to an outer wall of the first hollow tube.

7. The catheter of claim 1, wherein the distal end of the first hollow tube includes one or more openings permitting fluid communication with the lumen of the first hollow tube through the distal end.

8. The catheter of claim 1, wherein the distal end of the first hollow tube includes a single opening permitting the passage of tools from within the lumen beyond the distal end of the first hollow tube.

9. A method of positioning a catheter within a chamber of a heart of a patient, the method comprising the steps of:
providing a shunt in place within the heart and a guide snare including a distal end, the distal end releasably anchored to the shunt within the heart, the guide snare extending out of the heart and a proximal end of the guide snare exiting the patient via an insertion site in a blood vessel;
attaching a catheter to the guide snare, the catheter including a first hollow tube and a second hollow tube, the second hollow tube adapted to fit about the guide snare;
advancing the catheter along the guide snare through the insertion site into the patient's blood vessel and into the heart chamber.

10. The method of claim 9, wherein the second hollow tube of the catheter includes a distal end, a proximal end and an opening through the second hollow tube connecting the distal end and the proximal end, and catheter is attached to the guide snare by inserted the proximal end of the guide snare into the distal end of the second hollow tube.

11. The method of claim 9, wherein the second hollow tube of the catheter includes a distal end, a proximal end, an opening through the second hollow tube connecting the distal end and the proximal end, and a lengthwise split through a wall of the second hollow tube from distal end to proximal end, the split normally closed, the second hollow tube made of material sufficiently deformable which will allow the guide snare to be inserted through the split into the opening of the second hollow tube and sufficiently resilient for the split to close after the guide snare is inserted, and the catheter is attached to the guide snare by inserted the guide snare through the split into the opening.

12. A medical device comprising a first elongate instrument and a guide tube:
the first elongate instrument having a distal end, a proximal end, a length and a lumen connecting the distal end to the proximal end;
the guide tube having a distal end, a proximal end, a length and a lumen connecting the distal end to the proximal end, the distal end and the proximal end each including an opening into the lumen;
the length of the guide tube being substantially less then the length of the first elongate instrument;
the guide tube being attached to the first elongate instrument such that the distal end of the guide tube is proximate the distal end of the first elongate instrument; and
the openings and lumen of the guide tube sized to receive a guide snare.

13. A catheter comprising a first hollow tube and a guide ring:
the first hollow tube having a distal end, a proximal end, a length and a lumen connecting the distal end to the proximal end;
the guide ring having a opening for receiving a guide snare such that the guide snare extends essentially parallel to the to the length of the first hollow tube;
the guide ring being attached to the first hollow tube proximate the distal end of the first hollow tube; and
the guide ring including a split to allow a guide snare to be inserting within the guide ring and releasably retaining the guide snare within the guide ring.
